(19) United States
(12) Patent Application Publication
(10) Pub. No.: US 2016/0120511 A1
(43) Pub. Date: May 5, 2016

(54) ULTRASONIC MEDICAL DEVICE

(71) Applicants: THE UNIVERSITY OF TOKYO, Tokyo (JP); HITACHI ALOKA MEDICAL LTD.), Mitaka-shi, Tokyo (JP)

(72) Inventors: Takashi Azuma, Tokyo (JP); Akira Sasaki, Tokyo (JP); Ryosuke Aoyagi, Tokyo (JP); Shu Takagi, Tokyo (JP); Kazunori Itani, Mitaka-shi (JP); Keisuke Fujiwara, Mitaka-shi (JP); Hideki Takeuchi, Mitaka-shi (JP)

(73) Assignees: THE UNIVERSITY OF TOKYO, Tokyo (JP); HITACHI ALOKA MEDICAL, LTD., Mitaka-shi, Tokyo (JP)

(21) Appl. No.: 14/891,732

(22) PCT Filed: May 27, 2014
(86) PCT No.: PCT/JP2014/063997

§ 371 Date: Nov. 17, 2015

(30) Foreign Application Priority Data
May 30, 2013 (JP) 2013609

(51) Int. Cl. A61B 8/08; A61N 7/02

(52) U.S. Cl. CPC A61B 8/485; A61B 8/085; A61N 7/02

(57) ABSTRACT

A modulation frequency control unit (36) controls a displacement-use transmission unit (34) such that displacement-use ultrasonic beam (EB) is subjected to modulation processing using a relatively high modulation frequency and a relatively low modulation frequency. A displacement measurement unit (24) measures the displacement of a tissue in a treatment area (P) at each of the modulation frequencies, and a coagulation measurement unit (25) measures local coagulation in the treatment area (P) on the basis of the measurement result of the displacement at the relatively high modulation frequency, and measures wide-area coagulation in the treatment area (P) on the basis of the measurement result of the displacement at the relatively low modulation frequency. Consequently, for example, the presence or absence of local coagulation immediately after coagulation has occurred, and the like, can be measured with high accuracy, and further, for example, the size of wide-area coagulation after coagulation has progressed, and the like, can be measured with high accuracy.

### TECHNICAL FIELD

The present invention relates to an ultrasound medical apparatus that measures coagulation of a tissue.

### BACKGROUND ART

A treatment method is known in which a high intensity focused ultrasound (HIFU) is irradiated onto, for example, a living body, and a treatment site such as a tumor is heated and coagulated using acoustic energy of the ultrasound.

It is known that when a tissue is heated and coagulated, an elastic modulus (Young's modulus) of the tissue is increased after the coagulation. In addition, because a relatively intense ultrasound such as HIFU generates a radiation force in the direction of travel of the ultrasound, for example, a displacement of about 10-100 μm (micrometers) can be caused at a tissue at a focal point site of the HIFU ultrasound beam.

Because of this, it is possible to cause a displacement at the tissue with a relatively intense ultrasound such as HIFU and measure a reduction of the displacement due to the increase in the elastic modulus, and to consequently observe the coagulation of the tissue. For example, by modulating the amplitude of the HIFU ultrasound wave with a modulation frequency of f_{M} to vary the intensity of the radiation force, a vibration may be excited at the tissue of the focal point site, and a displacement or a rate of the vibration may be measured by an ultrasound diagnostic apparatus.

A method of detecting the coagulation of the tissue using this principle and mapping a detection result thereof on an image is known as HMI (Harmonic Motion Imaging) (refer to Patent Documents 1 and 2). Because the frequency of the radiation force and the vibration of the tissue is twice the modulation frequency f_{M}, this method is called HMI.

### RELATED ART REFERENCES

### Patent Documents

Patent Document 1: US2005/0004466 A

Patent Document 2: US2007/0276242 A

### DISCLOSURE OF INVENTION

### Technical Problem

In the treatment using the HIFU, it is desirable to suitably control HIFU according to the status of the coagulation at the treatment site. This is because, in the irradiation of HIFU to the living body, the attenuation and the influence of the phase distortion on the focal point sound pressure vary due to the difference in the acoustic characteristics of a propagation route of the HIFU depending on individual patients, and because it is difficult to optimize parameters for treatment in advance due to a change in the energy necessary for coagulation caused by a difference in cooling effect corresponding to a difference in bloodstream near the focal point. For example, if a time when the coagulation is started at the treatment site or a time when the coagulation is completed to a target size can be known, the HIFU irradiation can be controlled according to these times or the like.

In such circumstances, the present inventors have researched and developed for a technique for measuring coagulation of the tissue using ultrasound.

The present invention has been made as a result of this research and development, and an advantage thereof is that a measurement precision is improved in the measurement of coagulation of the tissue using ultrasound.

### Solution to Problem

According to one aspect of the present invention, there is provided an ultrasound medical apparatus comprising: a displacement wave processor that forms a displacement ultrasound beam and causes displacement of a tissue at a site of interest; a measurement wave processor that forms a measurement ultrasound beam and obtains a reception signal from the site of interest; a modulation controller that controls a modulation process for the displacement ultrasound beam; a displacement measurement unit that measures a displacement of a tissue at the site of interest based on a reception signal obtained through the measurement ultrasound beam; and a coagulation measurement unit that measures a coagulation of a tissue at the site of interest based on a measurement result of the displacement, wherein the modulation controller controls the displacement wave processor to apply a modulation process to the displacement ultrasound beam using a relatively high modulation frequency and a relatively low modulation frequency, the displacement measurement unit measures a displacement of the tissue at the site of interest for each of the modulation frequencies, and the coagulation measurement unit measures a local coagulation at the site of interest, based on a measurement result of the displacement with the relatively high modulation frequency, and measures coagulation of a wide area at the site of interest based on a measurement result of the displacement with the relatively low modulation frequency.

In the above-described configuration, the measurement ultrasound beam is, for example, a diagnostic ultrasound beam in a typical ultrasound diagnostic apparatus, and can be formed using a diagnostic ultrasound transducer. On the other hand, the displacement ultrasound beam is formed by a relatively high intensity ultrasound in a degree to displace the tissue by the radiation force. The displacement ultrasound beam has a higher intensity compared to the diagnostic ultrasound beam, and may be formed by, for example, high intensity focused ultrasound (HIFU). Further, the tissue may be heated and coagulated by the high intensity focused ultrasound (HIFU). In this case, for example, the treatment site which is the target of treatment by the heating is the site of interest.

According to the above-described configuration, the tissue can be displaced in a limited manner in a relatively narrow region by the relatively high modulation frequency. As the region of displacement becomes narrower, more minute (local) coagulation can be detected. Because of this, with the relatively high modulation frequency, for example, the presence or absence of local coagulation immediately after the occurrence of the coagulation, and the time of the coagulation, can be measured with high precision. In addition, with the relatively low modulation frequency, the tissue can be displaced in a relatively wide region. As the region of displacement is widened, a larger (over a wider area) coagulation can be detected. Because of this, with the relatively low modulation frequency, for example, the size of the wide-area coagulation after progression and the completion timing of the treatment by the heating can be measured with high precision.

The relatively high modulation frequency and the relatively low modulation frequency are not limited to two modulation frequencies. For example, three or more modulation frequencies which differ from each other may be used, the most local coagulation may be measured by the highest modulation frequency, and the range of the coagulation to be measured may gradually become wider (wider area) as the modulation frequency is reduced. In addition, in the measurement of the coagulation, in addition to the presence or absence of the coagulation and the size of the coagulation (size) the degree of coagulation (distortion and hardness of the tissue) may be measured.

Preferably, the coagulation measurement unit measures a size of coagulation at the site of interest based on a measurement result of the displacement obtained for each of the modulation frequencies.

Preferably, the ultrasound medical apparatus further comprises a treatment wave processor that forms a treatment ultrasound beam, and heats and treats a tissue of the site of interest, wherein, when the coagulation measurement unit measures a size of the coagulation at the site of interest for each time phase over a plurality of time phases within a period of the heating, the coagulation measurement unit measures a size of a local coagulation at a time phase of an initial stage of occurrence of coagulation based on a measurement result of the displacement with the relatively high modulation frequency, and measures a size of coagulation of a wide area at a time phase after progression of the coagulation based on a measurement result of the displacement with the relatively low modulation frequency.

Preferably, the ultrasound medical apparatus further comprises an image formation unit that forms a coagulation state image in which a plurality of time phases are represented on one axis and a size of coagulation measured for each time phase is represented on the other axis.

Preferably, the displacement wave processor forms a displacement ultrasound beam in which a modulation process with the relatively high modulation frequency and a modulation process with the relatively low modulation frequency are combined, and the displacement measurement unit measures the displacement of the tissue at the site of interest for each of the modulation frequencies by extracting a frequency component corresponding to each of the modulation frequencies from a reception signal obtained through the measurement ultrasound beam.

Preferably, the displacement wave processor forms displacement ultrasound beam to which a modulation process is applied with the relatively high modulation frequency and a displacement ultrasound beam to which a modulation process is applied with the relatively low modulation frequency at time phases that are different from each other, the measurement wave processor forms a measurement ultrasound beam at a time phase corresponding to the modulation frequency for each of the modulation frequencies, and the displacement measurement unit measures the displacement of the tissue at the site of interest for each of the modulation frequencies based on the reception signal obtained through the measurement ultrasound beam formed for each of the modulation frequencies.

Preferably, the modulation controller controls the displacement wave processor to switch the modulation frequency from the relatively high modulation frequency to the relatively low modulation frequency when a size of coagulation measured based on a measurement result of the displacement with the relatively high modulation frequency reaches a threshold.

### Advantageous Effects

According to various aspects of the present invention, the measurement precision can be improved in the measurement of coagulation of the tissue using ultrasound. For example, according to a preferable configuration of the present invention, with a relatively high modulation frequency, for example, presence or absence of a local coagulation immediately after occurrence or the like can be measured with high precision, and, with a relative low modulation frequency, for example, a size of a wide-area coagulation after progression can be measured with high precision.

In addition, for example, according to a preferable configuration of the present invention, because it becomes possible to know a start time of coagulation, it is possible to correct non-ideal effects, such as an amount of attenuation from a body surface to the focal point and uneven effect of the acoustic characteristic on the propagation route, which differ for each patient when the energy which is input from the outside forms a sound pressure peak at the focal point, and to use as data for correcting effects having different temperature increase values even if a certain sound pressure peak is formed at the focal point due to an amount of absorption of the ultrasound at the focal point, the thermal characteristics, and individual differences in the amount of bloodstream.

### BRIEF DESCRIPTION OF DRAWINGS

FIG. 1 is a diagram showing an overall structure of an ultrasound medical apparatus according to a preferred embodiment of the present invention.

FIG. 2 is a timing chart showing an operation of the ultrasound medical apparatus of FIG. 1.

FIG. 3 is a diagram for explaining a vibration of a tissue by ultrasound for generating displacement, to which a modulation process is applied.

FIG. 4 is a diagram for explaining a relationship between a modulation frequency and coagulation.

FIG. 5 is a diagram showing an experimental result of a coagulation size detected for each modulation frequency.

FIG. 6 is a diagram for explaining a specific example setting of modulation frequencies.

FIG. 7 is a diagram showing collection of data from phase 1 to phase 13.

FIG. 8 is a flowchart of a specific example 1 which uses a combined wave of a plurality of modulation frequencies.

FIG. 9 is a diagram showing a modulation method having no DC component and a modulation method having a DC component.

FIG. 10 is a diagram showing a correspondence relationship between NH and NL in a modulation having no DC component.

FIG. 11 is a diagram showing two-modulation frequencies when NL=1 and NH=5.

FIG. 12 is a diagram showing two-modulation frequencies when NL=1 and NH=4.

FIG. 13 is a diagram showing a correspondence relationship between NH and NL in a modulation having a DC component.

FIG. 14 is a flowchart showing a specific example 2 in which a plurality of modulation frequencies are switched.

FIG. 15 is a flowchart showing a specific example 3 in which a plurality of modulation frequencies are stepwise changed.

FIG. 16 is a flowchart showing a specific example 4 in which the modulation frequency is switched based on a determination result of coagulation.

FIG. 17 is a diagram showing a correspondence relationship between a coagulation size and a modulation frequency.

FIG. 18 is a diagram showing a specific example of coagulation state image formed by the ultrasound medical apparatus of FIG. 1.

### EMBODIMENT

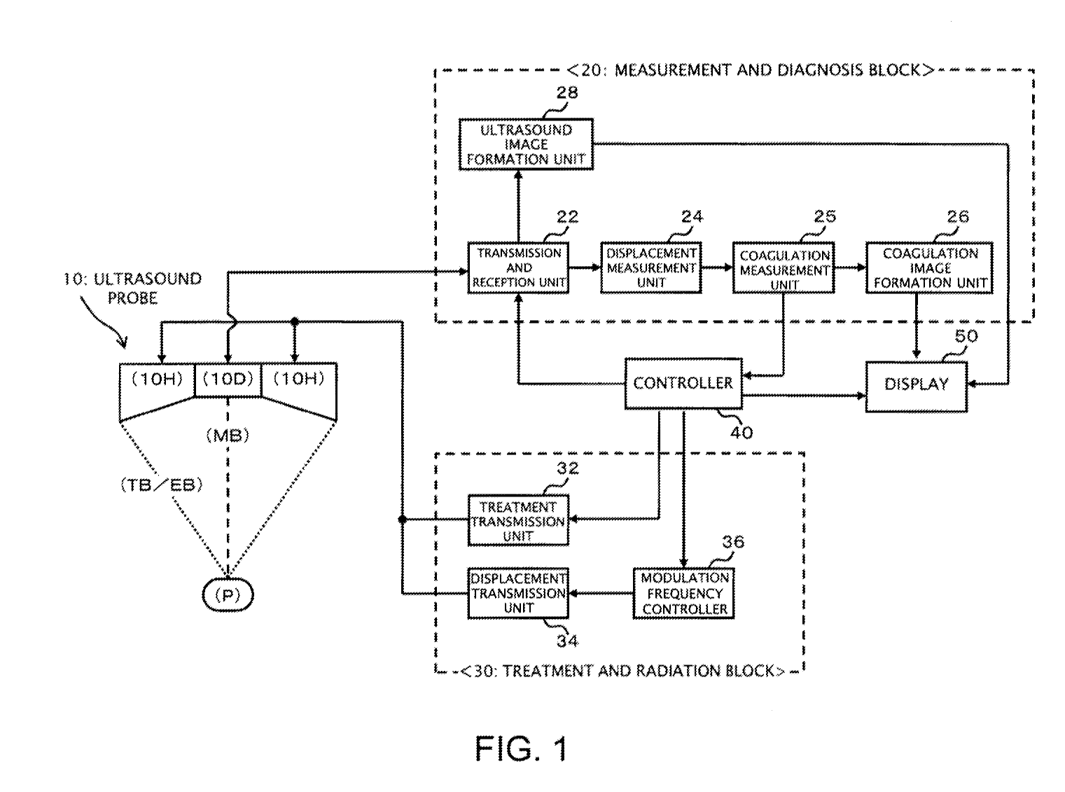

FIG. 1 is an overall structural diagram of an ultrasound medical apparatus according to a preferred embodiment of the present invention (“present ultrasound medical apparatus”). The present ultrasound medical apparatus comprises an ultrasound probe 10, which comprises a HIFU transducer 10H and a diagnostic transducer 10D.

The HIFU transducer 10H is a transducer which transmits the high intensity focused ultrasound (HIFU) and comprises, for example, a plurality of transducer element arranged two-dimensionally. The HIFU transducer 10H is used to form a treatment ultrasound can TB and transmit the high intensity focused ultrasound to a treatment site P such as, for example, cancer cells or a tumor, and to heat and treat the treatment site P.

The HIFU transducer 10H also forms a displacement ultrasound beam EB and transmits the ultrasound for generating displacement to the treatment site P, to generate a radiation force at the treatment site P and displace the tissue. The displacement ultrasound beam EB is a beam which is formed at an intensity to generate an effective radiation force at the treatment site P, and, for example, the treatment ultrasound beam TB may be used as the displacement ultrasound beam EB.

On the other hand, the diagnostic transducer 10D comprises, for example, a plurality of transducer elements arranged two-dimensionally, and transmits and receives relatively weak ultrasound for forming an ultrasound image to and from a subject (patient) having the treatment site P, for example. In other words, the transducer 10D transmits and receives ultrasound of an intensity (energy) similar to that of known, typical ultrasound diagnostic apparatuses.

The diagnostic transducer in also forms a measurement ultrasound beam MB and transmits the measurement ultrasound to the treatment site P, and obtains a reception signal along the measurement ultrasound beam MB. The reception signal obtained along the measurement ultrasound beam MB is used for measurement of displacement at the treatment site P by the radiation force of the displacement ultrasound beam EB.

The ultrasound probe 10 has the inner surface recessed in a shape of, for example, a bowl as a transducer plane. For example, the diagnostic transducer 10d is provided at the bottom portion positioned at the center of the inside, which is recessed in the bowl shape, and the HIFU transducer 10H is provided surrounding the diagnostic transducer 10D. The shape of the transducer plane of the ultrasound probe 10 is not limited to the bowl shape, and is desirably a shape appropriate to, for example, the treatment usage or the like. Here, all of the transducer elements or some of the transducer elements are used for both usages of HIFU and diagnosis.

A measurement and diagnosis block 20 includes a transmission and reception unit 22 which controls transmission and reception. of the diagnostic transducer 10D. The transmission and reception unit 22 outputs a transmission signal corresponding to each of the plurality of transducer elements of the diagnostic transducer 10D, to control the diagnostic transducer 10D and form a transmission beam, and further, applies a phased summation process or the like to the reception signal obtained from each of the plurality of transducer elements to obtain the reception signal along the reception beam.

The transmission and reception unit 22 scans the diagnostic ultrasound beam in a three-dimensional space including the treatment site P or within a cross section, to collect reception signals for an image. Further, an ultrasound image formation unit 28 forms image data for a three-dimensional ultrasound image or a two-dimensional tomographic image based on the collected reception signal, and an ultrasound image corresponding to the image data is displayed on a display 50.

A user (inspector) checks the position of the treatment site P or the like from the ultrasound image displayed on the display 50, and inputs positional information of the treatment site P in the present ultrasound medical apparatus using an operation device or the like (not shown). Alternatively, a configuration may be employed in which the present ultrasound :medical apparatus checks the position of the treatment site P by an image analysis process on the ultrasound image or the like, to obtain the positional information.

In addition, the transmission and reception unit 22 controls the diagnostic transducer 10D to form the measurement ultrasound beam MB, and obtains the reception signal along the measurement ultrasound beam MB. The displacement measurement unit 24 measures the displacement at the treatment site P based on the reception. signal obtained along the measurement ultrasound beam MB. Further, a coagulation measurement unit 25 measures coagulation of the tissue at the treatment site P based on a measurement result of the displacement at the treatment site P. Further, a coagulation image formation unit 26 forms a coagulation state image based on a measurement result of the coagulation at the treatment site P, and the coagulation state image is displayed on the display 50. The processes at the displacement measurement unit 24, the coagulation measurement unit 25, and the coagulation image formation unit 26 will be described later in detail.

A treatment and radiation block 30 includes a treatment transmission unit 32. The treatment transmission unit 32 outputs a transmission signal corresponding to each of the plurality of transducer elements of the HIFU transducer 10H, to control the HIFU transducer 10H and form the treatment ultrasound beam TB. The treatment transmission unit 32 is controlled by a controller 40, and, for example, a treatment ultrasound beam TB is formed in which the focal point is set within the treatment site P.

The treatment and radiation block 30 also includes displacement transmission unit 34. The displacement transmission unit 34 outputs a transmission signal corresponding to each of the plurality of transducer elements of the HIFU transducer 10H, to control the HIFU transducer 10H and form the displacement ultrasound beam EB. A modulation process is applied to the displacement ultrasound beam EB, where a modulation frequency of the modulation process is controlled by a modulation frequency controller 36. The modulation frequency controller 36 is controlled by the controller 40.

When the high intensity focused ultrasound (HIFU) is transmitted along the treatment ultrasound beam TB and the treatment site P is thus heated, the tissue at the treatment site P is coagulated. It is known that the elastic modulus of the tissue (Young's modulus) increases after the coagulation. In order to know the change of the elastic modulus of the tissue, the present ultrasound medical apparatus transmits ultrasound along the displacement ultrasound beam EB to generate a radiation force, and measures a displacement of the tissue at the treatment site P due to the radiation force. The measurement of the displacement is executed based on a reception signal obtained along the measurement ultrasound beam MB.

Each unit in the measurement and diagnosis block 20 and each unit in the treatment and radiation block 30 can be realized, for example, using hardware such as a processor and an electronic circuit. The controller 40 is formed from, for example, hardware having a calculation function and software (program) that defines an operation of the hardware. The display 50 is, for example, a liquid crystal display or the like.

Alternatively, the measurement and diagnosis block 20 may be realized by a typical ultrasound diagnostic apparatus. Thus, the present medical apparatus may be realized by a system in which an ultrasound treatment apparatus corresponding to the treatment and radiation block 30 and an ultrasound diagnostic apparatus corresponding to the measurement and diagnosis block 20 are combined.

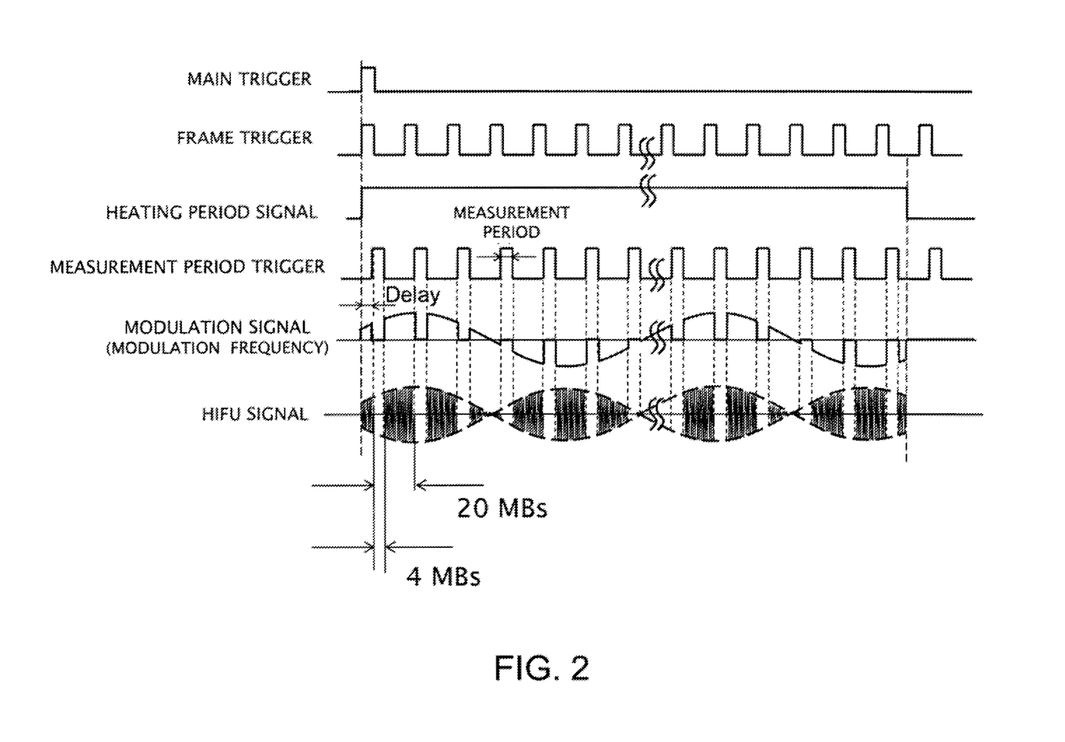

FIG. 2 is a timing chart showing an operation of the ultrasound medical apparatus of FIG. 1 (present ultrasound medical apparatus). For the portion (structure) shown in FIG. 1, reference numerals of FIG. 1 will be used in the following description.

A main trigger is a signal showing a start timing of treatment by the high intensity focused ultrasound (HIFU), and is output from the controller 40 to each unit in the present ultrasound medical apparatus in response to a treatment start operation by the user (inspector).

A frame trigger is a signal indicating a frame start of the measurement ultrasound beam MB. The transmission and reception unit 22 sequentially forms a plurality of measurement ultrasound beams MB toward the treatment site P from, for example, the time of rising of the frame trigger. For example, between two frame triggers, 10 transmission beams are formed toward the treatment site P, and two reception beams for each transmission beam, and a total of 20 reception beams, are formed. The numbers of transmission beams and the reception beams are not limited to the above-described specific numbers.

A heating period signal is a signal indicating a heating process period of the treatment site P by the treatment ultrasound beam TB, and in a period from rise to fall of the heating period signal, for example, the treatment ultrasound beam TB is formed with the treatment site P as the focal point.

A measurement period trigger is a signal indicating a period of displacement measurement, and a period from rise to fall of the measurement period trigger is the measurement period of the displacement.

A modulation signal is a modulation signal used in the modulation process of the treatment ultrasound beam TB, and is output, for example, from the modulation frequency controller 36 to the displacement transmission unit 34.

A HIFU signal is a transmission signal of the treatment ultrasound beam TB, and is obtained by, for example, the displacement transmission unit 34 applying amplitude modulation to a continuous wave of a frequency of about 2 MHz according to the modulation signal.

The modulation signal is set to have an amplitude of 0 (zero) in the measurement period from the rise to the fall of the measurement period trigger, and as a result, the amplitude of the HIFU signal is also set to 0 during the measurement period, and the transmission of the treatment ultrasound beam TB is stopped during the measurement period.

The measurement period trigger is output with a delay (Delay) with respect to the frame trigger. The delay is suitably adjusted, for example, by the user. With this configuration, of the plurality of measurement ultrasound beams MB (for example, 20 reception beams) formed between two frame triggers, one or a number of measurement ultrasound beams MB which fit in the measurement period can be selectively used for the measurement.

In the present ultrasound medical apparatus, the displacement ultrasound beam ED that causes displacement is amplitude-modulated with the modulation frequency f_{M}, and the intensity of the radiation force is varied, so that a vibration is excited in the tissue of the focal point site and the displacement of the vibration is measured using the measurement ultrasound beam MB. During this process, the modulation frequency f_{M} of the displacement ultrasound beam EB is controlled by the modulation frequency controller 36. Alternatively, in place of the amplitude modulation, a frequency modulation with the modulation frequency f_{M} may be employed.

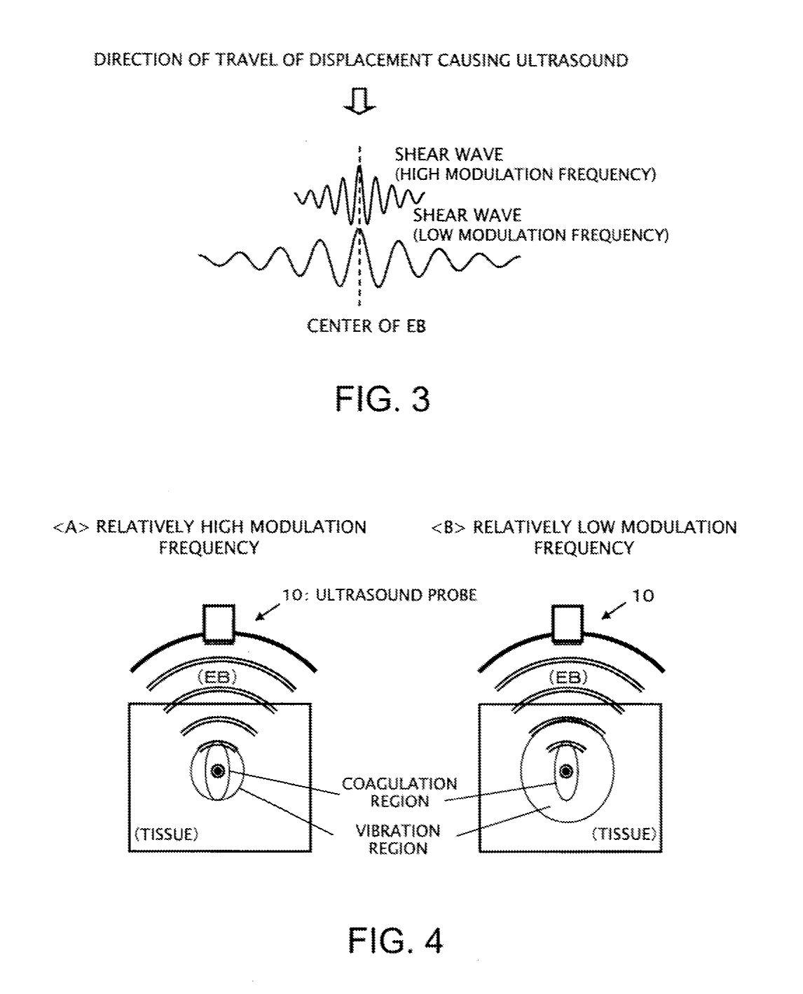

FIG. 3 is a diagram for explaining vibration of the tissue by the ultrasound for causing displacement to which the modulation process is applied. When the displacement ultrasound beam EB is formed along the direction of travel (up-down direction) shown by an arrow in FIG. 3, and the ultrasound is irradiated, a lateral wave called a shear wave is generated which travels in the tissue from the center of the displacement ultrasound beam EB in both left and right directions. A frequency of vibration of the shear wave is twice the modulation frequency f_{M} of the displacement causing ultrasound.

FIG. 3 shows waveforms of a shear wave generated by ultrasound of a relatively high modulation frequency and a shear wave generated by ultrasound of a relatively low modulation frequency. With a higher modulation frequency, the vibration site of the tissue would exist in a localized manner, and thus, the position resolution at the displacement measurement is increased and such a configuration is suited for detection of a minute coagulation area. However, when the modulation frequency is high, the region of displacement is narrow. Therefore, the high modulation frequency is not suited for detection of coagulation over a wide area exceeding the region of the displacement, and a low modulation frequency is desirable for detection of coagulation over a wide area.

FIG. 4 is a diagram for explaining a relationship between the modulation frequency and the coagulation. FIG. 4 schematically shows the displacement ultrasound beam EB radiated from the ultrasound probe 10 and the coagulation region and the vibration region in the tissue.

<A> shows a case where a modulation process with a relatively high modulation frequency (for example, about 200 Hz) is applied on the displacement ultrasound beam EB. With the relatively high modulation frequency, the vibration region is relatively small and local, a change of an average elastic modulus of the tissue in the local vibration region is high, and thus, the relatively high modulation frequency is suited for detection of a small coagulation region.

On the other hand, <B> shows a case where a modulation process with a relatively low modulation frequency (for example, about 30 Hz) is applied on the displacement ultrasound beam EB. At the relatively low modulation frequency, the vibration region is relatively large, and is a wide area, and a change of an average elastic modulus of the tissue in the vibration region of a wide area can be measured, and thus, the relatively low modulation frequency is suited for detection of a large coagulation region.

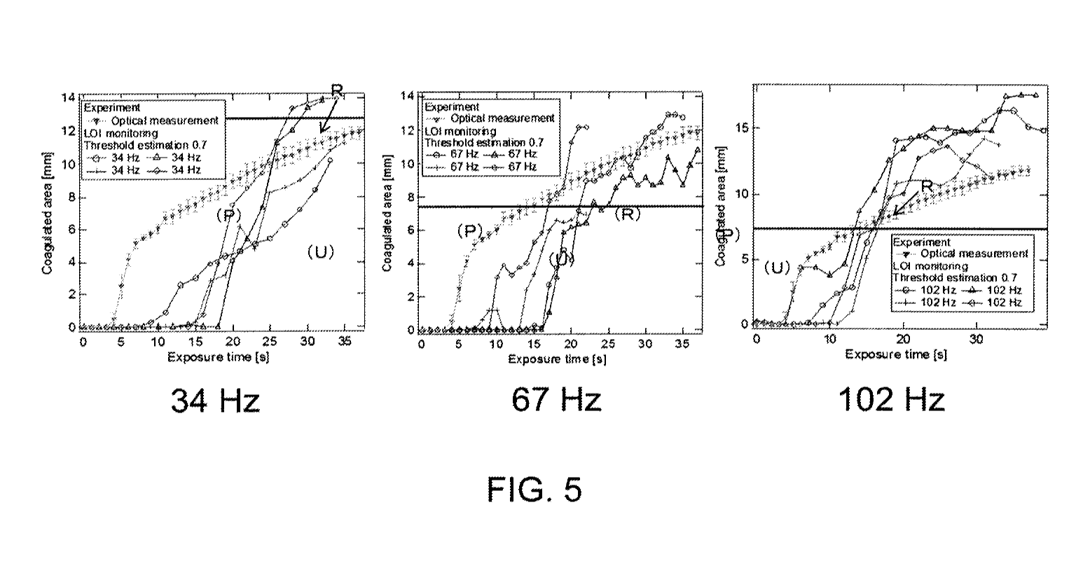

FIG. 5 is a diagram showing an experimental result of a coagulation size detected for each modulation frequency. FIG. 5 shows three experimental results for modulation frequencies of 34 Hz, 67 Hz, and 102 Hz, respectively, with the horizontal axis of each experimental result representing a time period in which the measurement site is heated, and the vertical axis representing the coagulation size which is measured. In each experimental result, a measurement result U obtained a plurality of times using the measurement ultrasound beam MB and an optical measurement result P are shown. The optical measurement result P is used as a referential value for the actual coagulation size.

As shown by the measurement result U obtained a plurality of times using the measurement ultrasound beam MB, the start of coagulation is detected at a time of 10-18 seconds for the modulation frequency of 34 Hz, at a time of 10-15 seconds for the modulation frequency of 67 Hz, and at a time of 4-10 seconds for the modulation frequency of 102 Hz. In other words, as the modulation frequency becomes higher, the more likely it is that the start of coagulation will be appropriately detected. In particular, for the modulation frequency of 102 Hz, the start of the coagulation is detected at almost the same time as the optical measurement result P serving as the referential value for the actual coagulation size.

A straight line H shown in each experimental result shows a range (vibration range) where the vibration (displacement) is caused, and the vibration range is about 13 mm for the modulation frequency of 34 Hz, about 8 mm for the modulation frequency of 67 Hz, and about 7 mm for the modulation frequency of 102 Hz. The vibration range shows a region where the measurement result of the coagulation size at a lower part of the straight line R is reliable, and as the modulation frequency is reduced, it becomes more suited for detection of coagulation of a wider area.

In the present ultrasound medical apparatus, the displacement ultrasound beam EB is modulation-processed using a relatively high modulation frequency and a relatively low modulation frequency. With the relatively high modulation frequency, presence or absence of local coagulation immediately after occurrence or the like is measured with high precision, and with the relatively low modulation frequency, the size of the coagulation of wide area after progression is measured with high precision. Therefore, in the present ultrasound medical apparatus, at least two modulation frequencies are utilized, and in the present ultrasound medical apparatus, for example, the modulation frequency is selected by the following method while maintaining the frame rate constant.

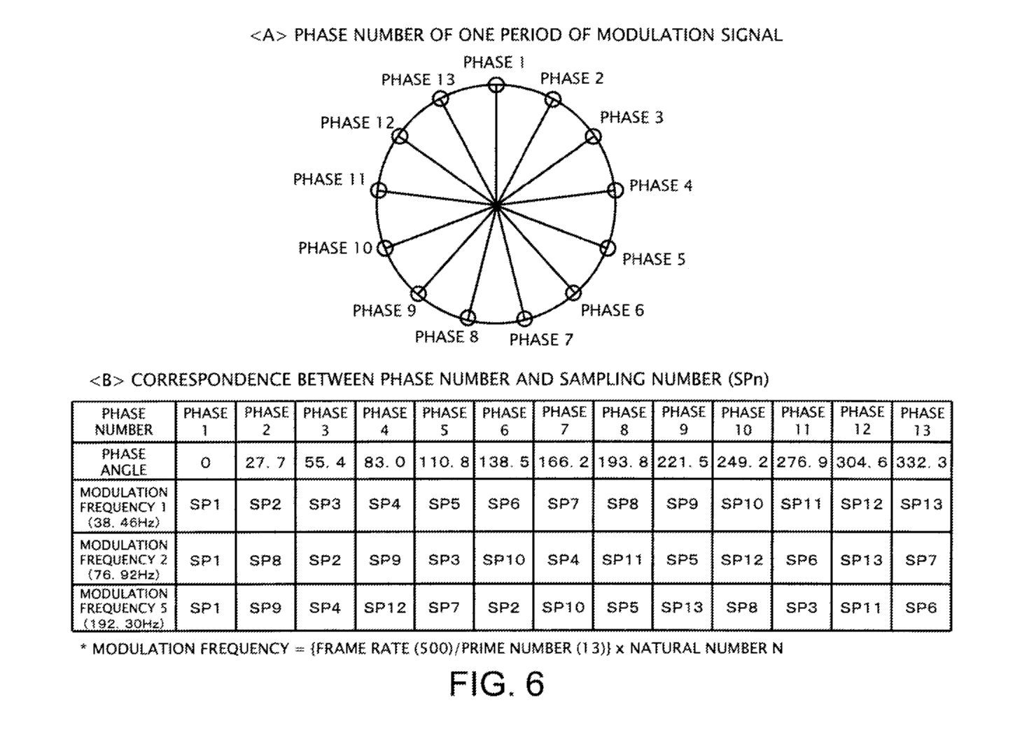

FIG. 6 is a diagram for explaining a specific example of setting of the modulation frequency. In the present ultrasound medical apparatus, the modulation frequency is determined by the following equation.

Modulation frequency (Hz)=(frame rate (Hz)/prime number)×natural number N (Equation 1)

The frame rate (Hz) and the prime number in Equation 1 are suitably set, for example, according to the specification of the apparatus, a treatment target, or the like. In the following description, a specific example will be described in which the frame rate is set to 500 Hz and the prime number is set to 13.

When the frame rate is 500 Hz and the prime number is 13, the modulation frequencies (Hz) obtained by Equation 1 are, if the natural number N is to be used as an identification number of the modulation frequency (modulation frequency N), modulation frequency 1 (38.46 Hz), modulation frequency 2 (76.92 Hz), . . . modulation frequency 5 (192.30 Hz), . . . .

The frame rate is a period of the frame trigger (FIG. 2), and is a period in which the measurement of the displacement is repeatedly executed at the same position by the measurement ultrasound beam MB. In other words, the frame rate becomes a sampling rate for the measurement of the displacement.

In FIG. 6, <A> shows a phase of one period of the modulation signal, and phase numbers (phase 1-phase 13) correspond to phase positions (phase angles) when one period of the modulation signal is equally divided by the prime number, 13. <B> shows correspondence relationship between the phase number (phase 1-phase 13) and sampling numbers indicating the order of acquisition. of data (SP1-SP13) for each modulation frequency.

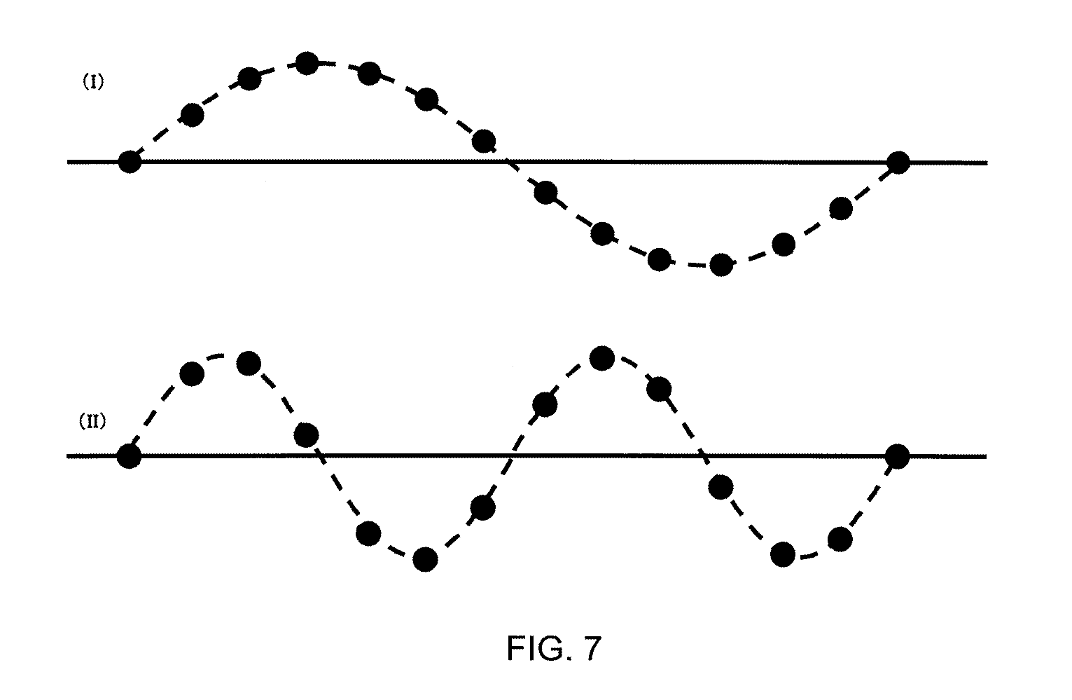

When the prime number is 13, based on Equation 1, the modulation frequency 1 (38.46 Hz) is 1/13^{th} of the frame rate (500 Hz). In other words, the sampling rate for the measurement of the displacement is 13 times the modulation frequency 1. Therefore, in the arrangement of the phase numbers shown in <A>, when data of the sampling number 1 (SP1) is obtained at phase 1, the data of the sampling number 2 (SP2) is obtained at phase 2, and subsequently, as shown in <B>, the data are obtained in the order of phase 3, phase 4, phase 5, . . . . Finally, the data of the sampling number 13 (SP13) is obtained at phase 13, and 13 data for one period are collected. This is shown in FIG. 7(I).

Referring again to FIG. 6, the modulation frequency 2 (76.92 Hz) is 2/13^{th} of the frame rate (500 Hz). That is, the sampling rate for the measurement of the displacement is 13/2 times the modulation frequency 2. Therefore, in the arrangement of the phase number shown in <A>, when the data of the sampling number 1 (SP1) is obtained at the phase 1, the data of the sampling number 2 (SP2) is obtained at phase 3, and, subsequently, as shown in <B>, the data are sequentially obtained in the order of phase 5, phase 7, phase 9, . . . . When the data of the sampling number 7 (SP7) is obtained at phase 13, the data of the sampling number 8 (SP8) is obtained at the phase 2 of the next period. Subsequently, as shown in <B>, the data are obtained in the order of phase 4, phase 6, phase 8, . . . , and the data of the sampling number 13 (SP13) is obtained at the phase 12. In other words, as shown in <B>, by obtaining the data of the sampling numbers 1-13 (SP1-SP13), the data of one period (two periods on the waveform) from phase 1 to phase 13 are collected. This is shown in FIG. 7(II).

Referring again to FIG. 6, the modulation frequency 5 (192.30 Hz) is 5/13^{th} of the frame rate (500 Hz). In other words, the sampling rate for the measurement of the displacement is 13/5 times the modulation frequency 5. Therefore, in the arrangement of the phase numbers shown in <A>, when data of the sampling number 1 (SP1) is obtained at phase 1, the data are obtained in order as shown in <B>, and, by obtaining data of the sampling numbers 1-13 (SP1-SP13), data of one period from phase 1 to phase 13 are collected.

Similarly, in other modulation frequencies that are not exemplified in FIG. 6 also, by obtaining data of sampling numbers 1-13 (SP1-SP13), the data of one period from phase 1 to phase 13 can be collected.

According to the specific example of the setting of the modulation frequency shown in FIG. 6, data without imbalance in the phase can be collected with relatively small sampling number (for example, 13), and the problem of aliasing can also be avoided. Because of the collection of data having no imbalance in the phase, the value of RMS (Root Mean Square) becomes a value which is not significantly deviated from the value of RMS when the data are collected with sufficiently fine sampling.

In the present ultrasound medical apparatus, the modulation. frequency is determined by the above-described Equation 1, the displacement ultrasound beam EB is modulation-processed using a relatively high modulation frequency and a relatively low modulation frequency, presence or absence of local coagulation. immediately after occurrence is measured with high precision by the relatively high modulation frequency, and the size of coagulation of a wide area after progression is measured with high precision by the relatively low modulation frequency. In other words, the present ultrasound medical apparatus uses a plurality of modulation frequencies including a relatively high modulation frequency and a relatively low modulation frequency. In the following, specific example configurations using a plurality of modulation frequencies will be described.

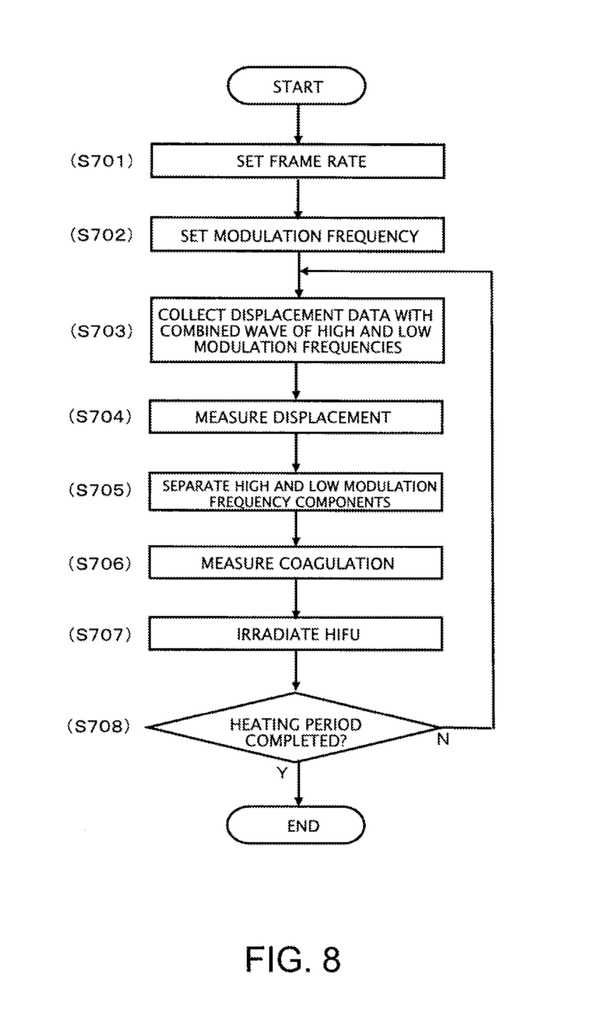

FIG. 8 is a flowchart showing a specific example 1 which uses a combined wave of a plurality of modulation frequencies. First, the frame rate is set (S701). For example, when a depth of the diagnosis range is 15 cm, a time required for ultrasound to travel back and force would be 15 (cm)×2/1500(m/s)=100 μs (microseconds). When the number of measurement ultrasound beams MB is 20, for example, the frame rate would be 1/(20×100 μm)=500 Hz. Of the 20 measurement ultrasound beams MB, for example, 4 measurement ultrasound beams MB are used. For example, as shown in FIG. 2, when there are 20 measurement ultrasound beams MB, and of these, 4 measurement ultrasound beams MB are used for the measurement, a ratio of time in which temperature increases to time in which the temperature decreases is 16:4, and thus, because the time of temperature increase is 4 times the time of temperature decrease, the temperature can be efficiently increased (in reality, depending on the phase of the modulation wave, the above-described ratio of increase to decrease may not be obtained, but when the overall treatment time is considered, the above-described relationship is mostly true).

Next, the modulation frequency is set (S702). By setting the period of the modulation to be sufficiently lower than the measurement period (refer to FIG. 2), the influence of the measurement period on the vibration can be reduced or avoided. For example, when the measurement period is 400 μs, the modulation frequency must be set sufficiently lower than 2.5 kHz. In addition, in order to obtain a sufficient sensitivity for a coagulation region of a size of a few mm to a few cm, it is desirable that a wavelength of the shear wave is shorter than about 10 cm. When a sonic speed of the shear wave (lateral wave) is 1 m/s, the modulation frequency in which the wavelength of the shear wave becomes less than or equal to 10 cm is a modulation frequency greater than or equal to 10 Hz. In addition, in order to secure a time resolution of the order of seconds, it is desirable to repeat a plurality of vibrations per second, and thus, the modulation frequency would be greater than or equal to a few Hz. While satisfying these conditions, and according to the specific example configuration explained with reference to FIG. 6, the relatively low modulation frequency (low modulation frequency) is set at 38.46 Hz, and the relatively high modulation frequency (high modulation frequency) is set at 192.30 Hz, for example.

A vibration is caused at the tissue of the treatment site P by the displacement ultrasound beam EB obtained by a modulation. process in which the high modulation frequency (192.30 Hz) and the low modulation frequency (38.46 Hz) are combined, and the data for displacement measurement is collected through the measurement ultrasound beam MB (S703). As explained with reference to FIG. 6, the data of sampling numbers 1-13 (SP1-SP13) are collected. While it is possible to obtain the data of one period of the modulation frequency by the data of one set of sampling numbers 1-13 alone, for example, in order to reduce or remove the influence of noise or the like, data of two sets are collected. Alternatively, data of greater than or equal to 2 sets may be collected.

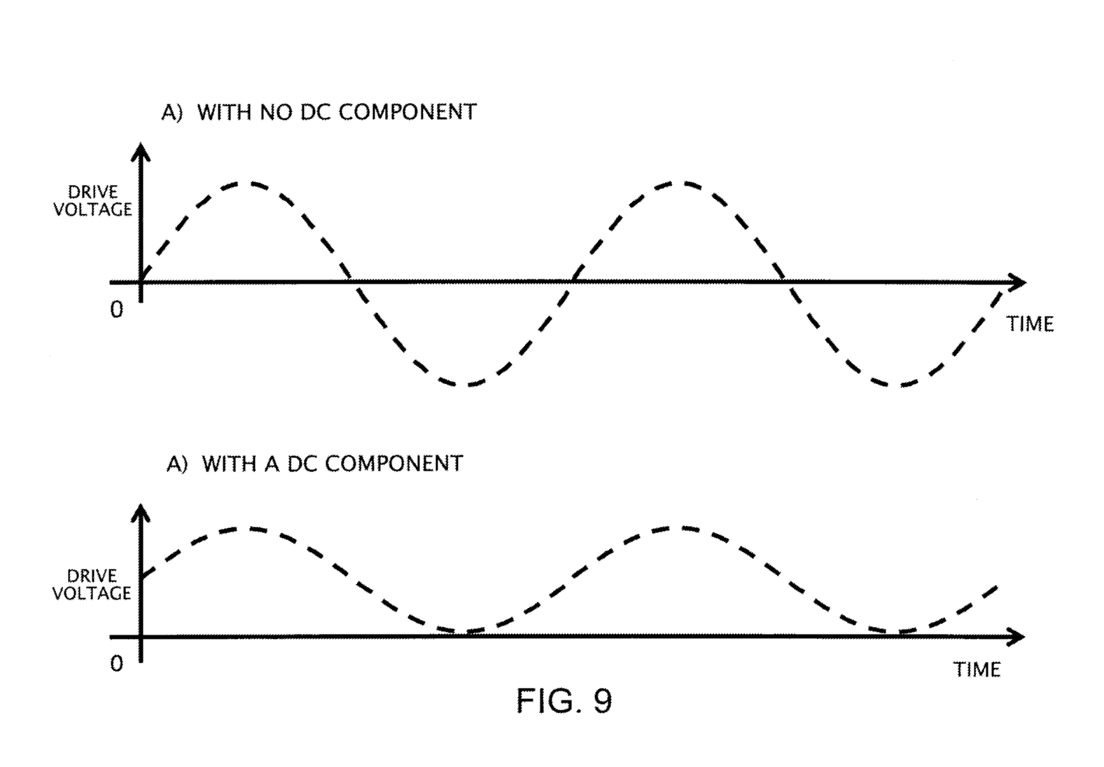

In the following description, the frame rate in relation to an example of a prime number of N will be referred to as FR, two modulation frequencies will be referred to as FL and FH (wherein FH>FL), and FR/M will be referred to as F1 (FR/M=F1). Further, FL/F1 will be referred to as NL (FL/F1=NL), and FH/F1 will be referred to as NH (FH/F1=NH). The radiation force itself is proportional to the square of the sound pressure. Thus, in consideration that a combined wave of two modulation frequencies is generated and that aliasing exists between these waves, the conditions of the modulation frequency that can be selected will now be sorted out. The method of modulation includes a modulation method having no DC component as shown in FIG. 9 and a modulation method having a DC component. As the frequency component of the radiation force appears in a different manner, these modulation methods will be separately described.

First, a case will be described in which modulation without a DC component is applied. The modulation frequencies of the drive waveform are NL and NH. The vibration components measured for these modulation frequencies include six vibration components of the radiation force; that is, 2NL, 2NH, DC, NH−NL, NH+NL, and NL−NH, and four aliasing components caused by sampling at M; that is, N−2NH, M−2NL, M−NM−NL, and M−NH+NL (the above-described DC is a difference frequency component of NL's or NH's. In addition, NL−NH is normally a negative number, but in consideration of the matching with the aliasing, this component must also be considered, and thus, is included herein). Of these components, only conditions where NH and NL can be distinctively and independently measured can be applied, and therefore, these components will be described.

As a pattern (1), as a condition where the vibration components of the radiation force may match, in consideration that NH>NL, or the like, a condition of NH−NL=2NL or NH=3NL would correspond to this case. For example, in the case of M=13, (NL, NH)=(1, 3), (2, 6), (3, 9), and (4, 12) satisfy the condition.

Next, as a pattern (2), for a condition where the vibration component of the radiation force matches the aliasing component (the condition for match of aliasing components would be similar to pattern (1)), in consideration that M is a prime number (that is, M is not a multiple of some other number), the following 5 conditions would correspond to this case:
when M−2NL=NH+NL, or M=3NL+NH; in the case of M=13, (NL, NH)=(1, 10), (2, 7), (3, 4), (5, 11), and (6, 8) would correspond to this case; the remaining two are twice M;
when M−2NL=NH−NL, or M=NL+NH; in the case of M=13, (NL, NH)=(1, 12), (2, 11), (3, 10), (4, 9), (5, 8), and (6, 7) would correspond to this case;
when M−2NH=NH+NL, or M=3NH+NL; in the case of M=13, (NL, NH)=(1, 4), (2, 8), and (3, 12) would correspond to this case; the remaining two match 2 times and 3 times M;
when M−2NH=NH−NL or M=3NH−NL; in the case of M=13, (NL, NH)=(2, 5) and (5, 6) would correspond to this case; and
when M−2NL=NL−NH or M=3NL−NH; in the case of M=13, (NL, NH)=(7, 8) and (8, 11) would correspond to this case.

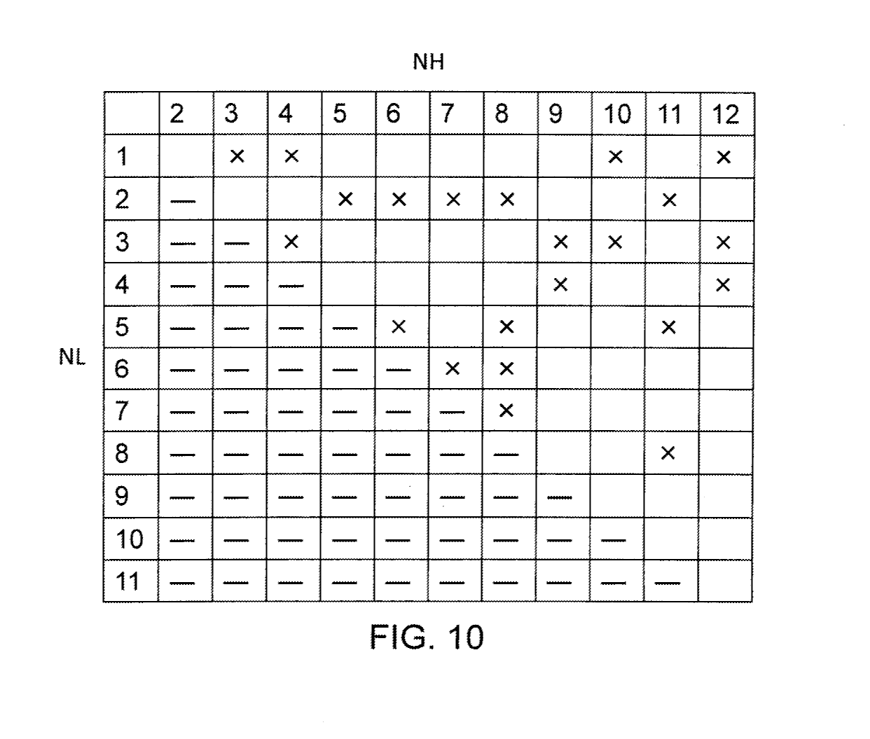

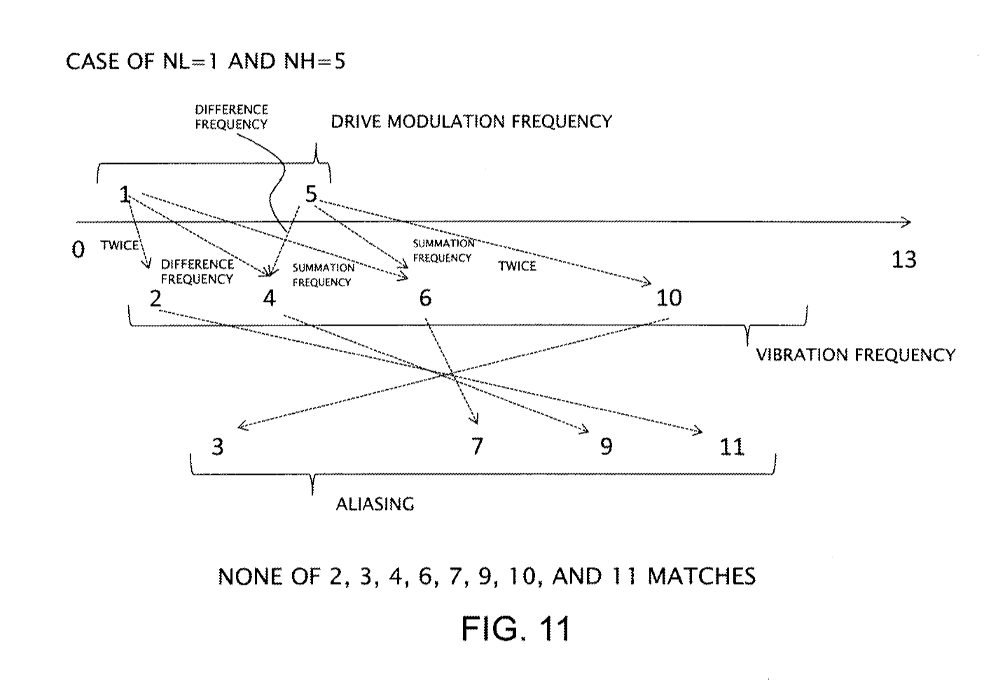

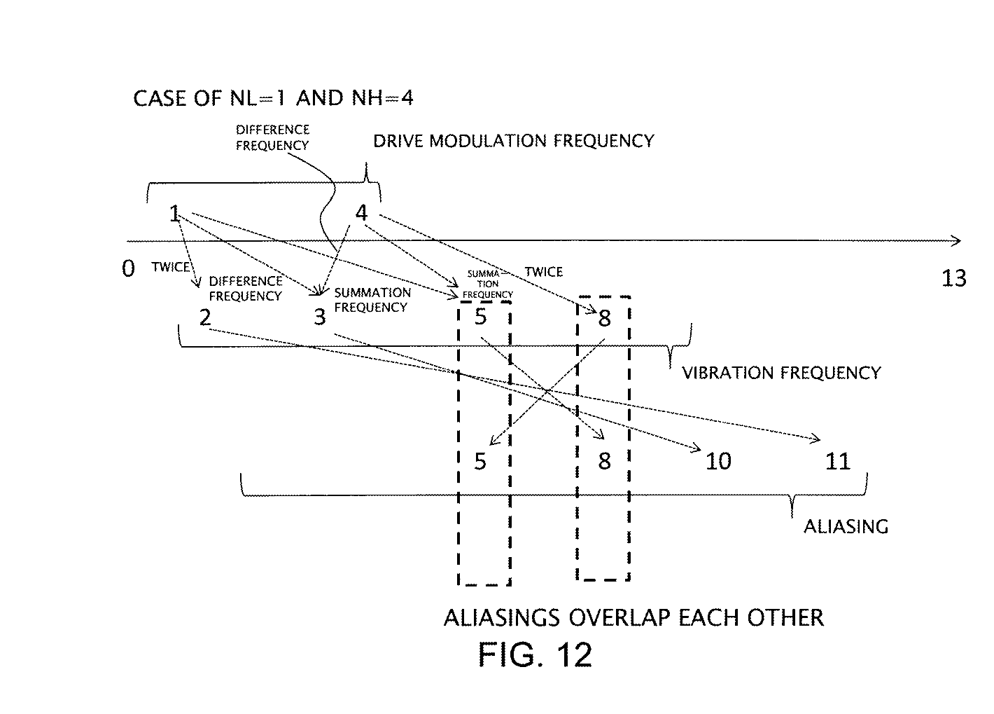

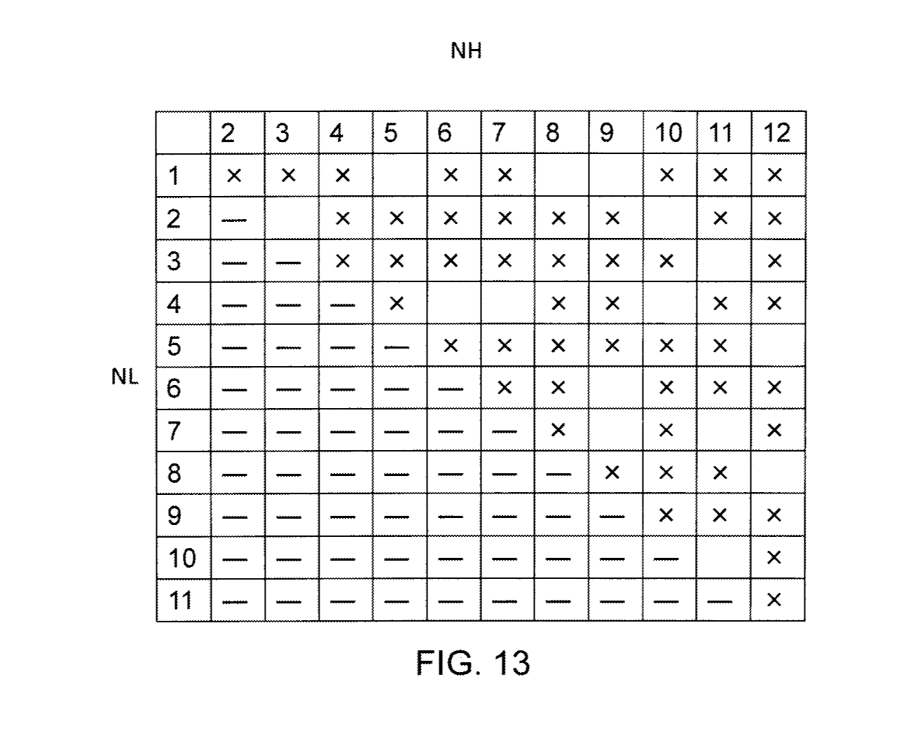

FIG. 10 summarizes these numbers in a table in the table shown in FIG. 10, it can be understood that because a configuration with a value of NH/NL close to 1 does not have a significant advantage with the use of the two modulation frequencies, the combinations of 1 and 5-9, 11 or 2 and 9, and 10, 12 or 3 and 8 or 11 are suitable as the two modulation frequencies. A typical example configuration is shown in FIGS. 11 and 12.

Next, a case where modulation with a DC component is applied will be described. The modulation frequencies of the drive waveform are similar to the above, and are NL and NH. The vibration components measured with respect to these modulation frequencies include eight vibration components of the radiation force; that is, NL, NH, 2NL, 2NH, 2NH, DC, NH−NL, NH+NL, and NL−NH, and seven aliasing components caused by sampling with M, that is, M−NH, M−NL, M−2NH, M−2NL, M−NM−NL, M−NH+NL, and M+NH−NL. Of these, only conditions where the NH and NL can be distinctively and independently measured can be applied, and thus, these conditions are considered.

As a pattern (1), for a condition where the vibration components of the radiation force match, in consideration of NH>NL or the like, the following corresponds to this condition:
when NH=2NL; in the case of M=13, (NL, NH)=(1, 2), (2, 4), (3, 6), (4, 8), (5, 10), and (6, 12) correspond to this condition; and
when NH−NL=2NL, or NH=3NL; in the case of M=13, (NL, NH)=(1, 3), (2, 6), (3, 9), and (4, 12) would correspond to this condition.

As a pattern (2), for a condition where the vibration component of the radiation force and the aliasing component match, as the calculation is similar to the case with no DC component, the detailed calculation will be omitted, and the following corresponds to this condition:
when M=NL+NH; in the case of M=13, (NL, NH)=(1, 12), (2, 11), (3, 10), (4, 9), (5, 8), and (6, 7) would correspond to this condition;
when M=2NL+NH; in the case of M=13, (NL, NH)=(1, 11), (2, 9), (3, 7), (4, 5), (7, 12), and (8, 10) would correspond to this condition; the remaining two match twice M;
when M=2NH+NL; in the case of M=13, (NL, NH)=(3, 5), (1, 6), (8, 9), (6, 10), (4, 11), and (2, 12) would correspond to this condition;
when M=2NH−NL; in the case of M=13, (NL, NH)=(1, 7), (3, 8), (5, 9), (7, 10), (9, 11), and (11, 12) would correspond to this condition;
when M=3NL+NH; in the case of M=13, (NL, NH)=(1, 10), (2, 7), (3, 4), (5, 11), (6, 8), and (9, 12) would correspond to this condition;
when M=3NL−NH; in the case of M=13, (NL, NH)=(7, 8) and (8, 11) would correspond to this condition;
when M=3NH+NL; in the case of M=13, (NL, NH)=(1, 4), (5, 7), (2, 8), (9, 10), (6, 11), and (3, 12) would correspond to this condition; and
when M=3NH−NL; in the case of M=13, (NL, NH)=(2, 5), (5, 6), and (10, 12) would correspond to this condition.

FIG. 13 shows these numbers summarized in a table. In the table shown in FIG. 13, because the configuration where NH/NL is close to 1 does not have a significant advantage of the use of the two modulation frequencies, it can be understood that 1 and 5, 8, or 9, 2 and 10, and 3 and 11, or the like are suited for the two modulation frequencies. In comparison between the configuration with the DC component and the configuration without the DC component, because the configuration without the DC component has more choices of the modulation frequencies, the configuration without the DC component is easier to use. However, because the DC component may be mixed due to non-linearity of the transmission amplifier or the like, when it is difficult to strictly set restrictions on the performances of the amplifier, it is desirable to select the configuration with the DC component.

Referring again to FIG. 8, when the displacement data collected at S703, the displacement is then measured based on the collected data (S704). The displacement is measured, for example, for each depth in a depth direction of the measurement ultrasound beam MB. In addition, for each depth, for example, adjacent data (n and n+1, where n=1, 2, . . . 25) within one data set including 26 points (26 time phases) are compared to each other by mutual correlation calculation or the like, and displacement is calculated for each depth. Alternatively, for example, the displacement may be calculated by a comparison between a reference time phase before the heating treatment and the most recent time phase. Alternatively, prior to the calculation of the displacement, a baseband removal process, a noise removal process, or the like may be executed as necessary.

Next, a high modulation frequency component and a low modulation frequency component are separated (S705). For example, a bandpass filter or the like is used to separate and extract a vibration component (displacement component) corresponding to the high modulation frequency (192.30 Hz) and a vibration component (displacement component) corresponding to the low modulation frequency (38.46 Hz). Alternatively, a configuration may be employed in which, prior to the measurement of the displacement at S704, a data component corresponding to the high modulation frequency and a data component corresponding to the low modulation frequency are separated, and the displacement is measured for each modulation frequency at S704. Here, the order of the separation of the two frequency components and the RMS is important. Because with RMS the data is squared once, a summation frequency or a difference frequency of the two frequencies occurs, which may cause aliasing, which would consequently further narrow the usable conditions shown in FIG. 13.

Next, coagulation is measured based on the vibration component obtained for each modulation frequency component (S706). For example, for each depth and for each modulation frequency component, from the vibration component (displacement component) obtained over two sets (two frames), a root mean square (RMS) of the displacement, that is, the effective value, is calculated, and the effective value is set, for example, as an amplitude of a current time phase (most recent time phase). Further, for each depth and for each modulation frequency component, for example, when the amplitude of the current time phase becomes 70% of the amplitude of the reference time phase before the heating treatment, it is judged that coagulation has started at the depth at the current time phase. In addition, for a plurality of depths for which it is judged that the coagulation has started at the current time phase, the size of the coagulation (coagulation size) is calculated from the range of the plurality of depths. The method of detecting coagulation for each depth (each position) and mapping the detection result on, for example, an image or the like is called LMI (Localized Motion Imaging).

Alternatively, in S706, in place of the effective value, the amplitude of the current time phase may be obtained using fitting or lock-in detection. When the fitting or the lock-in detection is used, the data is re-arranged prior to commencement. In other words, as explained above with reference to FIG. 6, for example, for the high modulation frequency (modulation frequency 5: 192.30 Hz), the acquisition order of the sampling number SPn, that is, the order of SP1, SP2, SP3, . . . SP13 and the order of the phase number do not match each other. Thus, the sampling numbers SPn are re-arranged so that the order is the order of the phase number, as shown in FIG. 6.

Then, the high intensity focused ultrasound (HIFU) is irradiated to the treatment site P (S707). For example, with the treatment ultrasound beam TB, HIFU is irradiated for about 0.5-1.0 seconds.

If the heating period (refer to FIG. 2) is not completed (S708), the measurement process of S703-S706 is executed in the next measurement period (refer to FIG. 2), and HIFU is irradiated in S707 after the measurement period. When the treatment ultrasound beam TB is used as the measurement ultrasound beam MB, HIFU in which the high modulation frequency and the low modulation frequency are combined is irradiated in S707, and the vibration component remaining after the HIFU irradiation is measured in S703-S706.

On the other hand, if the heating period (refer to FIG. 2) is completed (S708), the treatment at the treatment site P is completed. Alternatively, the treatment at the treatment site P may be completed when, in the measurement of coagulation in S706, a target size of coagulation is observed. Alternatively, a configuration may be employed in which, when the treatment at the treatment site P is completed, the treatment is applied for a treatment site P at a different position.

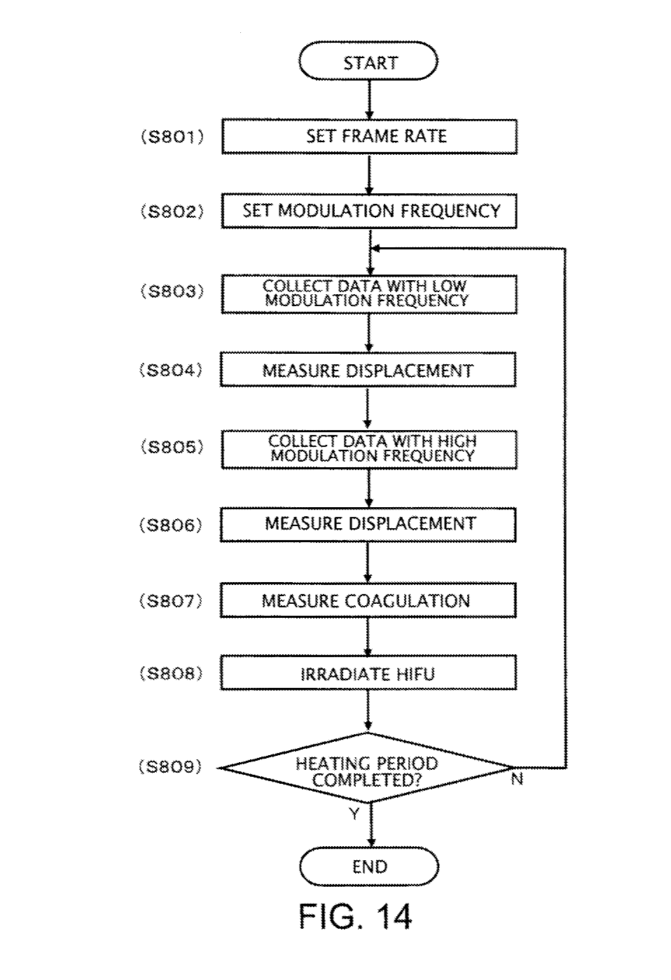

FIG. 14 is a flowchart showing a specific example 2 in which a plurality of modulation frequencies are switched. In the specific example 2 of FIG. 14 also, first, the frame rate is set (S801), and the modulation frequency is set (S802). The processes at S801 and S802 are identical to the processes at S701 and S702 of FIG. 8. Specifically, in S801, the frame rate is set to 500 Hz, and in S802, the low modulation frequency is set to 38.46 Hz and the high modulation frequency is set to 192.30 Hz.

In the specific example 2 of FIG. 14, for the low modulation frequency and the high modulation frequency, data is separately collected and the displacement is measured. Specifically, first, a vibration is caused at the tissue of the treatment site P by the displacement ultrasound beam EB obtained by the modulation process using the low modulation frequency, and the data for displacement measurement is collected through the measurement ultrasound beam MB (S803). For example, similar to S703 of FIG. 8, data of two sets are collected. Then, the displacement (vibration component) is measured based on the collected data (S804). For example, the displacement is calculated for each depth by a process similar to that in S704 of FIG. 8.

Next, a vibration is caused at the tissue of the treatment site P by the displacement ultrasound beam EB obtained by the modulation process using the high modulation frequency, and data for displacement measurement is collected through the measurement ultrasound beam MB (S805). For example, similar to S703 of FIG. 8, data of two sets are collected. Then, the displacement (vibration component) is measured based on the collected data (S806). For example, the displacement is calculated for each depth by a process similar to that in S704 of FIG. 8.

Then, coagulation is measured based on the vibration component obtained for each modulation frequency component (S807). For example, the depth where the coagulation has started at the current time phase is judged and the size of the coagulation (coagulation size) at the current time phase is calculated by a process similar to that in S706 of FIG. 8.

Next, the high intensity focused ultrasound (HIFU) is irradiated to the treatment site P (S808). For example, HIFU is irradiated by the treatment ultrasound beam TB for 0.5-1.0 seconds.

If the heating period (refer to FIG. 2) is not completed (S809), the measurement process of S803-S807 is executed in the next measurement period (refer to FIG. 2), and HIFU is irradiated at S808 after the measurement period. When the treatment ultrasound beam TB is used as the measurement ultrasound beam MB, the measurement process of S803 and S804 may be executed after irradiation of the HIFU which is modulation-processed by the low modulation frequency, and the measurement process of S805 and S806 may be executed after irradiation of HIFU which is modulation-processed by the high modulation frequency.

On the other hand, if the heating period (refer to FIG. 2) is completed (S809), the treatment at the treatment site P is completed. Alternatively, the treatment at the treatment site P may be completed when a target coagulation size is observed at the measurement of the coagulation at S807. Alternatively, a configuration may be employed in which, when the treatment at the treatment site P is completed, the treatment is executed at a treatment site P at a different position.

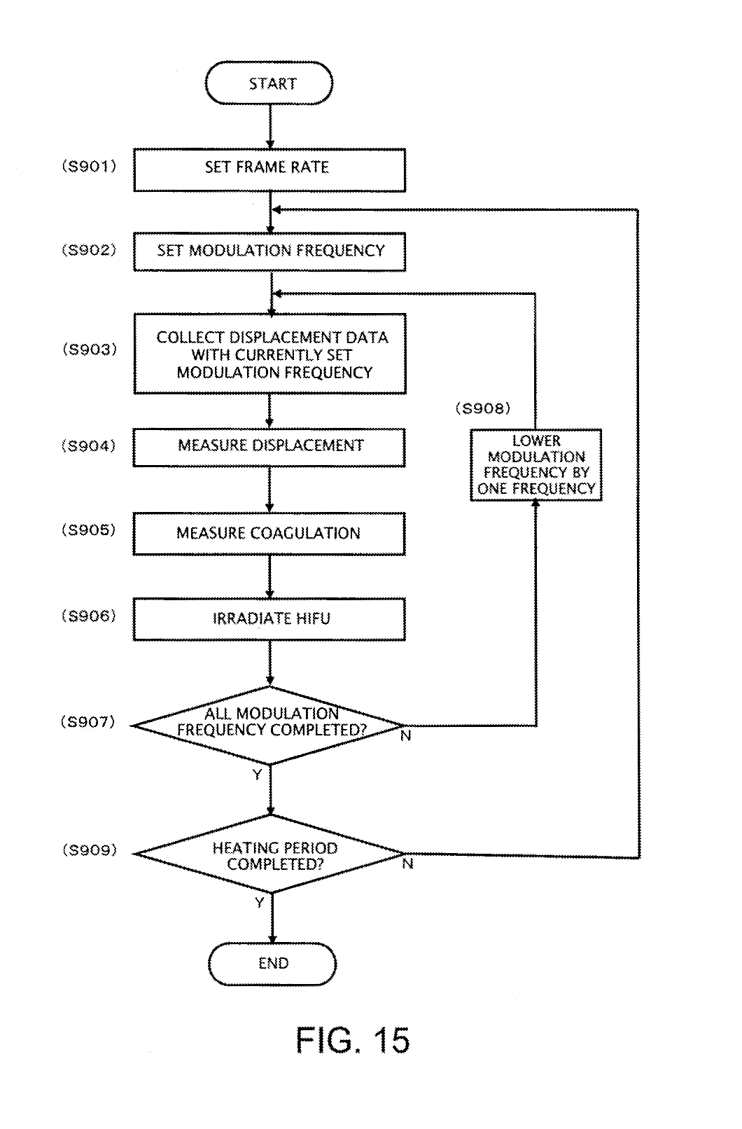

FIG. 15 is a flowchart showing a specific example 3 in which the plurality of modulation frequencies are stepwise changed. In the specific example 3 of FIG. 15 also, first, the frame rate is set (S901), and the modulation frequency is set (S902). The process at S901 is identical to the process at S701 of FIG. 8. That is, in S901, the frame rate is set to 500 Hz. In S902, a plurality of modulation frequencies are set by a process similar to the process at S702 of FIG. 8. For example, based on Equation 1, 6 modulation frequencies including modulation frequency 6 (230.76 Hz), modulation frequency 5 (192.30 Hz), modulation frequency 4 (153.84 Hz), modulation frequency 3 (115.28 Hz), modulation frequency 2 (76.92 Hz), and modulation frequency 1 (38.46 Hz) are set. In the initial state, the frequency is set at the modulation frequency 6.

A vibration is caused at the tissue of the treatment site P by the displacement ultrasound beam EB obtained by the modulation process using the modulation frequency which is currently set, and data for displacement measurement is collected through the measurement ultrasound beam MB (S903). For example, similar to S703 of FIG. 8, data of two sets are collected. Next, the displacement (vibration component) is measured based on the collected data (S904). For example, the displacement is calculated for each depth by a process similar to that of S704 of FIG. 8.

Further, coagulation is measured based on the vibration component obtained for the modulation frequency component which is currently set (S905). For example, the depth were the coagulation has started at the current time phase is judged or the size of the coagulation (coagulation size) at the current time phase is calculated by a process similar to that of S706 of FIG. 8.

Then, the high intensity focused ultrasound (HIFU) is irradiated to the treatment site P (S906). For example, the HIFU is irradiated by the treatment ultrasound beam TB for 0.5-1.0 second.

Next, it is checked whether or not processes related to all modulation frequencies have been completed (S907). In other words, it is checked whether or not all of the processes related to the six modulation frequencies have been completed, and if not, the modulation frequency is changed to a modulation frequency which is one frequency lower at S908, and the processes of S903-S906 are executed with the changed modulation frequency.

The processes of S903-S908 are repeatedly executed, and when completion of the processes related to all modulation frequencies are confirmed in S907, the process proceeds to S909.

If the heating period (refer to FIG. 2) is not completed (S909), the modulation frequency is set to the initial state, that is, the modulation frequency 6, at S902, and the processes of S903-S908 are executed. When the treatment ultrasound beam TB is used as the measurement ultrasound beam MB, in S906, the frequency is changed to a frequency which is one modulation frequency lower, and the HIFU which is modulation-processed by the changed modulation frequency is irradiated.

On the other hand, if the heating period (refer to FIG. 2) is completed (S909), the treatment at the treatment site P is completed. Alternatively, the treatment at the treatment site P may be completed when a target coagulation size is observed in the measurement of the coagulation at S905. Alternatively, a configuration may be employed in which, when the treatment at the treatment site P is completed, a treatment is executed for a treatment site P at a different position.

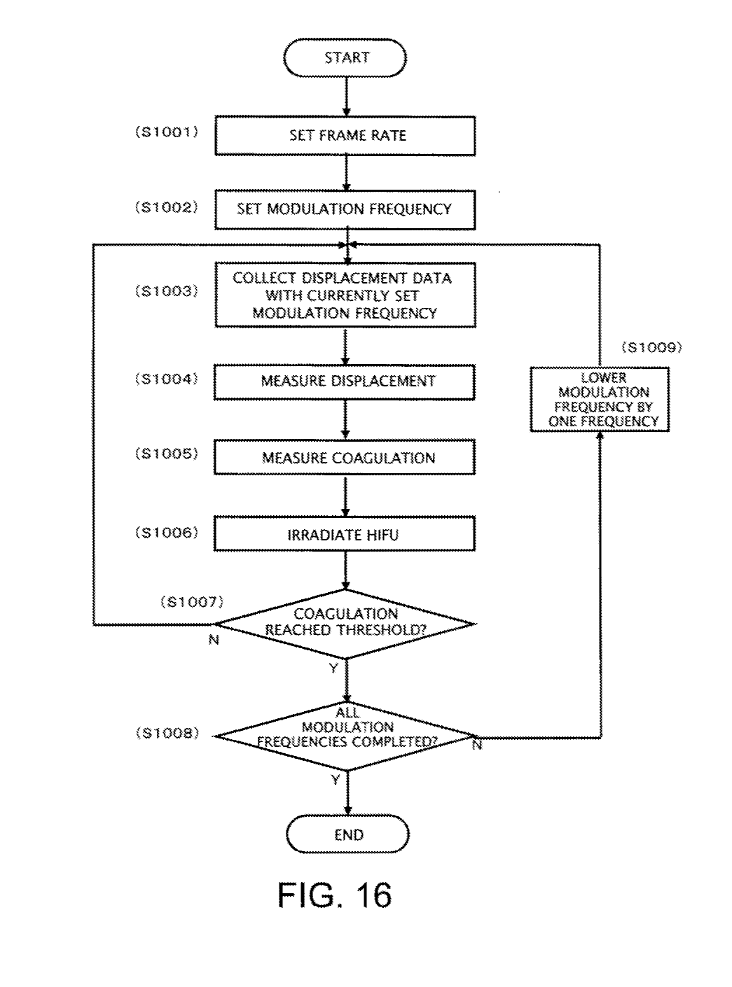

FIG. 16 is a flowchart showing a specific example 4 in which the modulation frequency is switched based on a determination result of coagulation. In the specific example 4 of FIG. 16 also, first, the frame rate is set (S1001), and the modulation frequencies are set (S1002). The process at S1001 is identical to the process at S701 of FIG. 8. In other words, in S1001, the frame rate is set to 500 Hz. In S1002, a plurality of modulation frequencies are set by a process similar to the process at S702 of FIG. 8. For example, based on Equation 1, 6 modulation frequencies including modulation frequency 6 (230.76 Hz), modulation frequency 5 (192.30 Hz), modulation frequency 4 (153.84 Hz), modulation frequency 3 (115.28 Hz), modulation frequency 2 (76.92 Hz), and modulation frequency 1 (38.46 Hz) are set. In the initial state, the frequency is set at the modulation frequency 6.

A vibration is caused at the tissue of the treatment site P by the displacement ultrasound beam EB obtained by the modulation process using the modulation frequency which is currently set, and data for displacement measurement is collected through the measurement ultrasound beam MB (S1003). For example, similar to S703 of FIG. 8, data of two sets are collected. Then, the displacement (vibration component) is measured based on the collected data (S1004). For example, the displacement is calculated for each depth by a process similar to that at S704 of FIG. 8.

Further, coagulation is measured based on the vibration component obtained by the modulation frequency which is currently set (S1005). For example, a depth where the coagulation has started at the current time phase is judged or the size of the coagulation (coagulation size) at the current time phase is calculated by a process similar to that in S706 of FIG. 8.

Then, the high intensity focused ultrasound (HIFU) is irradiated to the treatment site P (S1006). For example, the HIFU is irradiated by the treatment ultrasound beam TB for 0.5-1.0 second.

Then, for the modulation frequency which is currently set, it is judged whether or not the coagulation size has reached a threshold corresponding to the modulation frequency (S1007). In the specific example 4, a threshold related to the coagulation size is set for each modulation frequency.

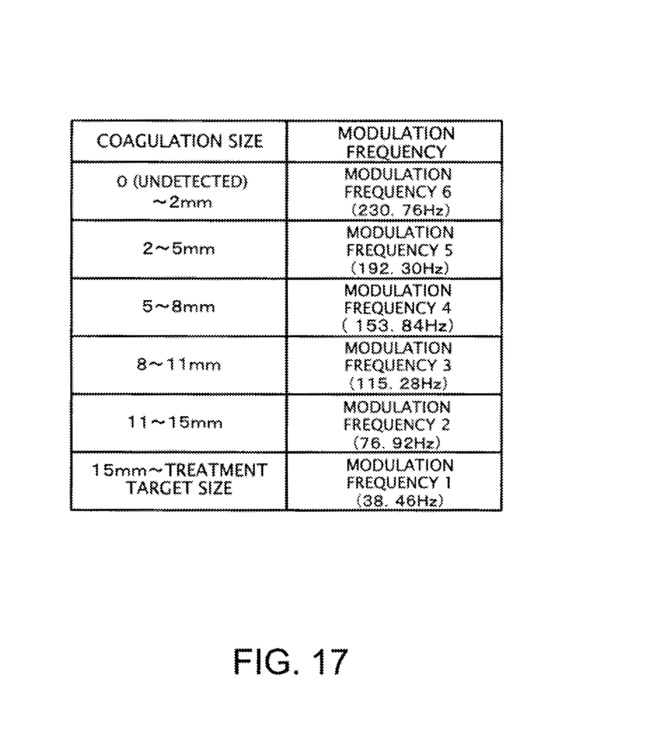

FIG. 17 is a diagram showing a correspondence relationship between the coagulation size and the modulation frequency. For example, in the range of the coagulation size from 0 (undetected) to 2 mm, the modulation frequency 6 (230.76 Hz) is used, and in a range of coagulation size of 2 mm to 5 mm, the modulation frequency 5 (192.30 Hz) is used. Other coagulation sizes are similarly handled as shown in FIG. 17.

Referring again to FIG. 16, in the measurement of coagulation using the modulation frequency 6 (230.76 Hz), the coagulation size of 2 mm is set as the threshold. When the coagulation size is greater than or equal to 2 mm or when the coagulation size becomes greater than 2 mm, in S1007, it is judged that the coagulation has reached the threshold.

When the coagulation has reached the threshold in S1007, it is checked whether or not the processes related to all modulation frequencies are completed (S1008). In other words, it is checked whether or not all of the processes related to the six modulation frequencies are completed, and if not, the modulation frequency is changed to one lower modulation frequency in S1009, and the processes of S1003-S1007 are executed with the changed modulation frequency.

In the measurement of the coagulation using the modulation frequency 5 (192.30 Hz), the coagulation size of 5 mm is set as the threshold (refer to FIG. 17), and in S1007, when the coagulation size is greater than or equal to 5 mm or when the coagulation size becomes greater than 5 mm, it is judged that the coagulation has reached the threshold.

In the measurement of the coagulation using the modulation. frequency 4 (153.84 Hz), the coagulation size of 8 mm is set as the threshold. In the measurement of the coagulation using the modulation frequency 3 (115.20 Hz), the coagulation size of 15 mm is set as the threshold. In the measurement of the coagulation using the modulation frequency 1 (38.46 Hz), a treatment target size is set as the threshold (refer to FIG. 17).

In this manner, the processes from S1003 to S1000 are repeated, measurement of the coagulation using the modulation frequency 1 (38.46 Hz) is executed, and when it is confirmed in S1007 that the treatment target size has been reached, it is confirmed in S1008 that all modulation frequencies are completed, and the treatment at the treatment site P is completed when the heating period (refer to FIG. 2) is completed also, the treatment at the treatment site P may be completed. Alternatively, a configuration may be employed in which, when the treatment at the treatment site P is completed, treatment is executed for a treatment site P at a different position.

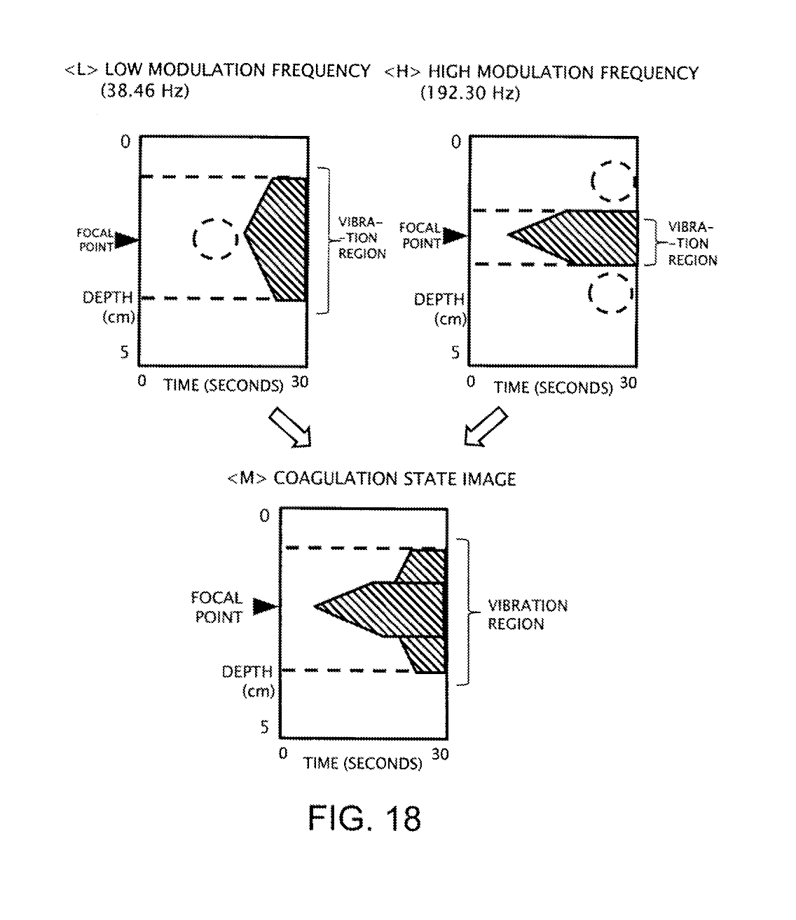

FIG. 18 is a diagram showing a specific example of coagulation state image formed by the ultrasound medical apparatus of FIG. 1 (present ultrasound medical apparatus). A coagulation image formation unit 26 forms a coagulation state image shown by <M> based on a measurement result of coagulation obtained from a coagulation measurement unit 25. In FIG. 18, the horizontal axis represents a heating time by the HIFU, and the vertical axis represents a depth. The focal point is the focal point of the treatment ultrasound beam TB that irradiates the HIFU. A portion shown by the slanted lines shows a region where coagulation is region confirmed.

<L> shows a measurement result of coagulation by the low modulation frequency (38.46 Hz). With the low modulation frequency, because the vibration region is relatively wide, the coagulation size that can be measured, that is, a widening in the vertical axis direction centered at the focal point, is relatively large. However, in <L>, the coagulation is not detected in the region shown by a dotted circle.

On the other hand, <H> shows a measurement result of coagulation by the high modulation frequency (192.30 Hz). With the high modulation frequency, the vibration region centered at the focal point is relatively small and local, a change of average elastic modulus of the tissue in the local vibration region is large, and thus, the configuration is suited for detection of a small coagulation at an early stage. Thus, with the high modulation frequency of <H>, the coagulation detected in a time corresponding to the region shown by the dotted circle in <L>. However, a portion exceeding the vibration region shown by the dotted circle in <H>, because no vibration is caused, the region is not suited for detection of the coagulation.

The coagulation image formation unit 26 forms the coagulation state image of <M> based on the measurement result <L> of the low modulation frequency and the measurement result <H> of the high modulation frequency, for example, by combining the measurement result <L> and the measurement result <H>.

In the coagulation state image of <M>, a small coagulation of an early stage is detected from the measurement result obtained based on the component of the high modulation frequency (192.30 Hz), and further, coagulation is detected over a relatively wide region from the measurement result obtained based on the component of the low modulation frequency (38.46 Hz).

Therefore, based on the coagulation state image of <M>, the user (inspector) can check the presence or absence of the local coagulation immediately after occurrence or the time of the coagulation, and moreover, can check, for example, the size of the coagulation after progression over the wide area.

When three or more modulation frequencies are used, the measurement results obtained from the three or more modulation frequencies are combined, to form the coagulation state image.

In the above description, a method of standardizing the displacement for each depth in one irradiation has been described. However, in reality, when a plurality of coagulation regions are irradiated in order, because of the influence of the existence of a site which is coagulated before the irradiation, there is a possibility that the displacement change due to coagulation cannot be sufficiently detected when the displacement is standardized for each depth within the irradiation. Specifically, when the coagulation region is set one-dimensionally as 1, 2, . . . , i, . . . , N, there is a possibility that the (i−1)th coagulation formation may affect the ith coagulation detection when the ith irradiation is executed. In consideration of this, it is also effective to measure a displacement distribution for the entirety of the irradiation. region before the coagulation, and to use the value of the displacement for the standardization.

In the above description, a method of causing modulation of the radiation force by modulating the amplitude has been described as a preferred embodiment of the present invention. In addition to this method, as another method of modulating the radiation force, there exists the following method. Namely, in this method, an amplitude of an enveloping line of a voltage applied to each element is set constant over the time axis, and the focal point of the beam is vibrated. In other words, if the propagation direction of the sound is the x direction and the y coordinate of the focal point position is fy, the apparatus is driven such that fy=fA sin(ωt). When fA is approximately equal to or greater than the beam width, the radiation force at each point would be considered as being modulated with an angular frequency of ω. For the method of amplitude-modulating the drive voltage, there exists a method of using a transformer in the transmission circuit, but in the method described in this paragraph, the drive voltage may be a constant and the transformer is not needed. As a result, the circuit size can be reduced, and there is a significant advantage in commercializing the apparatus. Therefore, it is also effective in practice to use the above-described method as the modulation method of radiation force described above.

In the description of the embodiment above, it has been described that the displacement is measured. However, from the viewpoint of detecting the change of hardness, a rate which is a derivative of displacement with respect to time, a distortion which is a derivative of displacement with respect to space, or a derivative of the distortion with respect to time may be measured, as these measurement targets can realize advantages similar to those when the displacement is measured. In addition, in the embodiment, an example configuration is explained which uses a prime number in the setting method of the modulation frequency, but in order to reduce the imbalance of the phases, the number does not need to be limited to a prime number. For example, when a number “14” is used in place of the prime number, advantages similar to those described in the description of the embodiment can be realized so long as “even numbers” and “7” are avoided as N.

A preferred embodiment of the present invention has been described. The above-described embodiment, however, is merely exemplary in all aspects, and does not limit the scope of the present invention. The present invention includes various modification within the scope and spirits of the invention. The treatment or the like using the ultrasound medical apparatus according to the present invention should be executed with sufficient caution under guidance of experts such as doctors.

### EXPLANATION OF REFERENCE NUMERALS

10 ULTRASOUND PROBE; 20 MEASUREMENT AND DIAGNOSIS BLOCK; 22 TRANSMISSION AND RECEPTION UNIT; 24 DISPLACEMENT MEASUREMENT UNIT; 25 COAGULATION MEASUREMENT UNIT; 26 COAGULATION IMAGE FORMATION UNIT; 28 ULTRASOUND IMAGE FORMATION UNIT; 30 TREATMENT AND RADIATION BLOCK; 32 TREATMENT TRANSMISSION UNIT; 34 DISPLACEMENT TRANSMISSION UNIT; 36, MODULATION FREQUENCY CONTROLLER; 40 CONTROLLER; 50 DISPLAY

1. An ultrasound medical apparatus comprising:
a displacement wave processor that forms a displacement ultrasound beam and causes displacement of a tissue at a site of interest;
a measurement wave processor that forms a measurement ultrasound beam and obtains a reception signal from the site of interest;
a modulation controller that controls a modulation process for the displacement ultrasound beam;
a displacement measurement unit that measures a displacement of a tissue at the site of interest based on a reception signal obtained through the measurement ultrasound beam; and
a coagulation measurement unit that measures a coagulation of a tissue at the site of interest based on a measurement result of the displacement, wherein
the modulation controller controls the displacement wave processor to apply a modulation process to the displacement ultrasound beam using a relatively high modulation frequency and a relatively low modulation frequency,
the displacement measurement unit measures a displacement of the tissue at the site of interest for each of the modulation frequencies, and
the coagulation measurement unit measures a local coagulation at the site of interest based on a measurement result of the displacement with the relatively high modulation frequency, and measures a coagulation of a wide area at the site of interest based on a measurement result of the displacement with the relatively low modulation frequency.

2. The ultrasound medical apparatus according to claim 1, wherein
the coagulation measurement unit measures a size of coagulation at the site of interest based on a measurement result of the displacement obtained for each of the modulation frequencies.

3. The ultrasound medical apparatus according to claim 2, further comprising:
a treatment wave processor that forms a treatment ultrasound beam, and heats and treats a tissue of the site of interest, wherein
when the coagulation measurement unit measures a size of the coagulation at the site of interest for each time phase over a plurality of time phases within a period of the heating, the coagulation measurement unit measures a size of a local coagulation at a time phase of an initial stage of occurrence of coagulation based on a measurement result of the displacement with the relatively high modulation frequency, and measures a size of coagulation of a wide area at a time phase after progression of the coagulation based on a measurement result of the displacement with the relatively low modulation frequency.

4. The ultrasound medical apparatus according to claim 3, further comprising:
an image formation unit that forms a coagulation state image in which a plurality of time phases are represented on one axis and a size of coagulation measured for each time phase is represented on the other axis.

5. The ultrasound medical apparatus according to claim 1, wherein
the displacement wave processor forms a displacement ultrasound beam in which a modulation process with the relatively high modulation frequency and a modulation process with the relatively low modulation frequency are combined, and
the displacement measurement unit measures the displacement of the tissue at the site of interest for each of the modulation frequencies by extracting a frequency component corresponding to each of the modulation frequencies from a reception signal obtained through the measurement ultrasound beam.

6. The ultrasound medical apparatus according to claim 1, wherein
the displacement wave processor forms a displacement ultrasound beam to which a modulation process is applied with the relatively high modulation frequency and a displacement ultrasound beam to which a modulation process is applied with the relatively low modulation frequency at time phases that are different from each other,
the measurement wave processor forms a measurement ultrasound beam at a time phase corresponding to the modulation frequency for each of the modulation frequencies, and
the displacement measurement unit measures the displacement of the tissue at the site of interest for each of the modulation frequencies based on the reception signal obtained through the measurement ultrasound beam formed for each of the modulation frequencies.

7. The ultrasound medical apparatus according to claim 1, wherein
the modulation controller controls the displacement wave processor to switch the modulation frequency from the relatively high modulation frequency to the relatively low modulation frequency when a size of coagulation measured based on a measurement result of the displacement with the relatively high modulation frequency reaches a threshold.

8. The ultrasound medical apparatus according to claim 3, wherein
the displacement wave processor forms a displacement ultrasound beam in which a modulation process with the relatively high modulation frequency and a modulation process with the relatively low modulation frequency are combined, and
the displacement measurement unit measures the displacement of the tissue at the site of interest for each of the modulation frequencies by extracting a frequency component corresponding to each of the modulation frequencies from a reception signal obtained through the measurement ultrasound beam.

9. The ultrasound medical apparatus according to claim 3, wherein
the displacement wave processor forms a displacement ultrasound beam to which a modulation process is applied with the relatively high modulation frequency and a displacement ultrasound beam to which a modulation process is applied with the relatively low modulation frequency at time phases that are different from each other,
the measurement wave processor forms a measurement ultrasound beam at a time phase corresponding to the modulation frequency for each of the modulation frequencies, and
the displacement measurement unit measures the displacement of the tissue at the site of interest for each of the modulation frequencies based on the reception signal obtained through the measurement ultrasound beam formed for each of the modulation frequencies.

10. The ultrasound medical apparatus according to claim 3, wherein
the modulation controller controls the displacement wave processor to switch the modulation frequency from the relatively high modulation frequency to the relatively low modulation frequency when a size of coagulation measured used on a measurement result of the displacement with the relatively high modulation. frequency reaches a threshold.

11. The ultrasound medical apparatus according to claim 1, wherein
the modulation controller determines the relatively high modulation frequency and the relatively low modulation frequency based on the following equation:
Modulation frequency (Hz))={frame rate (Hz)/prime number}×natural number N.

12. The ultrasound medical apparatus according to claim 11, wherein
the modulation controller determines the relatively high modulation frequency by setting the natural number N to a relatively large value, and determines the relatively low modulation frequency by setting the natural number N to a relatively small value.
